(19) United States
(12) Patent Application Publication
(10) Pub. No.: US 2012/0059211 A1
(43) Pub. Date: Mar. 8, 2012

(54) Method of diagnosing and treatment of hypertension

(76) Inventor: Ronald Weinstock, Fort Lauderdale, FL (US)

(21) Appl. No.: 13/199,568

(22) Filed: Sep. 2, 2011

Related U.S. Application Data
(63) Continuation-in-part of application No. 13/065,015, filed on Mar. 11, 2011.
(60) Provisional application No. 61/402,730, filed on Sep. 3, 2010.

(51) Int. Cl. A61N 2/04; A61N 2/02

(52) U.S. Cl. 600/14; 600/13

(57) ABSTRACT

A method for the provision of electrical, electromagnetic or magnetic stimulation to the T8 and T9 vertebrae and related off-shoot vertebrae of the human spine, through the use of probes, related induction coils and electrodes, imparting one or more of low frequency, high frequency, AC or magnetic fields and pulses, and their combinations, through the sympathetic and parasympathetic nervous systems, to diagnose and treat hypertension activity of the cells in any one or combination of the vascular wall, kidney or adrenal glands, to innervate and affect such cells to approximate normal function, inclusive of sodium regulation and regulated release of hormones from such cells of the adrenal gland as well as regulation of vascular tone through improved function of various ion channels and improved sympathic nervous system function.

### CROSS REFERENCE TO RELATED APPLICATIONS

This application claims the benefit under 35 USC 119(e) of the provisional patent application Ser. No. 61/402,730 filed Sep. 3, 2010, entitled Method of Diagnosing and Treatment of Hypertension, which is hereby incorporated by reference in its entirety; and is a continuation-in-part of application Ser. No. 13/065,015, filed Mar. 11, 2011, entitled EMF Probe Configurations for Electro-Modulation of Ionic Channels of Cells and Methods of Use Thereof, which is incorporated herewith in its entirety.

### FIELD OF THE INVENTION

The present invention relates to methods for regulating electrical movement of ions useful to the treatment of hypertension.

### BACKGROUND OF THE INVENTION

The role of biological ions as mediators of cellular activity is well established. Various technologies exist for controlling movement of ionic species across the membrane of living cell. Herein, the effectuation of such movement at a distance, using axonic pathways of the nervous system, is explored with specific reference to the spinal cord relative to the kidneys and adrenal glands as well as direct effect on these organs as well as a direct effect on the muscle cells in the vascular wall.

Prior art known to the inventor of an electrotherapeutic treatment of hypertension is reflected in U.S. Patent Application Publication U.S. 2007/0156201 to Rossing, entitled Hypertension Device and Method. The inventor's method and system differ in many respect from the work of Rossing.

Mandegar M, Remillard C V and Yuan J X have found that pulmonary arterial hypertension (PAH) is a hemodynamic abnormality that ultimately results in mortality due to right heart failure. Although the clinical manifestations of primary and secondary PAH are diverse, medial hypertrophy and arterial vasoconstriction are key components in the vascular remodeling leading to PAH. Abnormalities in the homeostasis of intracellular Ca(2+), transmembrane flux of ions, and membrane potential may play significant roles in the processes leading to pulmonary vascular remodeling. Decreased activity of K(+) channels causes membrane depolarization, leading to Ca(2+) influx. The elevated cytoplasmic Ca(2+) is a major trigger for pulmonary vasoconstriction and an important stimulus for vascular smooth muscle proliferation. Dysfunctional K(+) channels have also been linked to inhibition of normal apoptosis and contribute further to the medial hypertrophy.

The instant invention seeks to correct these dysfunctions through the use of electromagnetic and/or magnetic and/or electric fields and pulses.

### SUMMARY OF THE INVENTION

The present method relates to the provision of electrical, electromagnetic or magnetic stimulation to one or more of the T6 thru T12 and related off-shoot vertebrae of the human spine, through the use of probes, related induction coils and electrodes to impart one or more of low frequency, high frequency, AC, DC and combinations thereof, through the sympathetic and parasympathetic nervous systems, to diagnose and treat hypertension by the appropriate regulation of the activity of the cells in any one or combination of the vascular wall, kidney or adrenal glands, to innervate and affect such cells to approximate normal function, inclusive of sodium regulation and regulated release of hormones such as catacholamines aldosterone and cortisol from such cells of the adrenal gland as well as regulation of vascular tone through improved function of various calcium potassium and chloride ion channels and improved sympathic nervous system function.

An EMF probe assembly for the stimulation of T6 through T12 vertebrae and related neural offshoots, to diagnose and treat hypertension, the assembly comprises a probe at least one core formed of a ferro-metallic material positioned within said probe at least one induction coil wound around said at least one core; and an interface comprising a pad for contact of said probe with or near one or more of vertebrae T6 to T12 of their neural offshoots, and preferably the T8 and T9 vertebrae and their offshoots.

It is accordingly an object of the invention to provide an electromagnetic means of treatment of hypertension.

It is another object to regulate the activity of the cells in any one or combination of the vascular wall, kidney or adrenal gland, in order to reverse or preclude the onset of hypertension.

It is a further object of the invention to monitor selected electrical and/or electromagnetic wave patterns within the T6 to T12 and related neural off-shoots and vertebrae as well as areas at the sight of the kidney and adrenal glands to provide an early diagnosis, or diagnosis of, susceptibility to hypertension.

The above and yet other objects and advantages of the present invention will become apparent from the hereinafter set forth Brief Description of the Drawings and Detailed Description of the Invention.

### BRIEF DESCRIPTION OF THE DRAWINGS

FIG. 1 is a schematic view of the sympathetic and parasympathetic nervous systems and selected internal organs of the human body related thereto.

FIG. 2 is a flow diagram showing cytoplasmic calcium and other changes that occur when membrane potential changes are sensed by a cell.

FIG. 3 is a diagrammatic view showing the role that the various Ca2⁺′, K⁺ and Cl channels play in various causes of hypertension.

FIG. 4 is a graph showing the relationship between cell membrane potential, and calcium ion related current flow in a human cell.

FIG. 5 is a graph showing the relationship between cell membrane potential and concentration of free calcium ions within a cell.

FIG. 6 is a three-dimensional graph showing the relationship between cell membrane potential, calcium ion related current flow into the, cell and percent of time that calcium gated channels of the cell are open.

FIGS. 7 to 9 show diagnostic waveforms applied for cell treatment.

FIGS. 10 and 11 show electrical waveforms associated with a treatment of a first patient.

FIGS. 12 to 15 show electrical waveforms associated with treatment of a second patient.

FIGS. 16 and 17 show concepts for imagining of parameters relevant to normalization of cell function.

FIG. 18 is a schematic cross-sectional view of a vascular muscle cell.

FIG. 19 is a schematic view of the top of a smooth muscle cell, including a cross-section through an arteriole thereof showing ionic diffusion into the cell.

FIG. 20 is a side schematic view of an EMF probe assembly in accordance with the present invention.

FIG. 21 is a top plan view of the assembly of FIG. 20, taken along Line 21/24-21/24 of FIG. 20.

FIG. 22 is an enlarged schematic view of one of the inductive coil portions of the EMF probe assembly.

FIG. 23 is a schematic view of an alternative embodiment of the coil position of the assembly.

FIG. 24 is a top plan conceptual view taken along Line 21/24-21/24 of FIG. 20 showing the manner in which concentric electric fields associated with the B1 and B4 fields of the respective coils 102 and 112 produce electrical re-enforcement effects of E fields induced by the B fields.

FIG. 25 is a view, similar to that of FIG. 24, however showing the manner in which the induced electric fields E associated with the axial magnetic fields B1 and B8 of the respective coils cancels each other if current is reversed through coil 112, reversing axial magnetic field B4.

FIG. 26 is a view, similar to FIG. 6, however showing a complete treatment unit consisting of substantially identical upper and lower probes to those described in connection with said FIG. 6.

FIG. 27 is a view, similar to FIG. 26, however showing more details of the magnetic and electrical fields associated with the respective probes.

### DETAILED DESCRIPTION OF THE INVENTION

As is well-known, the sympathetic nervous system (SNS) is a branch of the autonomic nervous system along and of the central nervous system (CNS) and is also related to the parasympathetic nervous system (PNS).

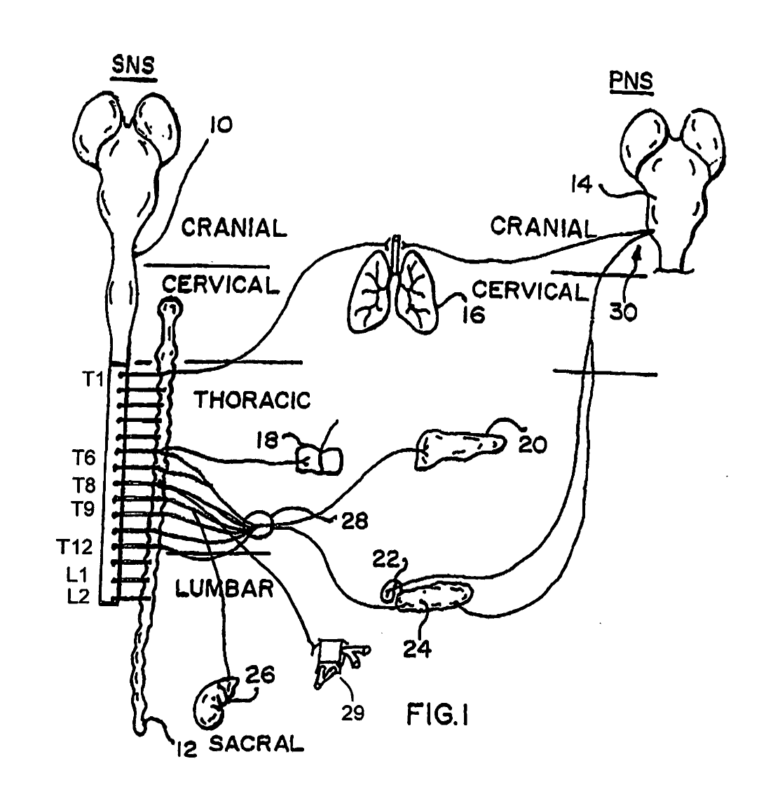

The SNS is active at a so-called basal level and becomes active during times of stress. As such, this stress response is termed the fight-or-flight response. The SNS operates through a series of interconnected neurons. Sympathetic neurons are frequently considered part of the PNS, although many lie within the CNS. Sympathetic neurons of the spinal cord are of course part of the CNS, and communicate with peripheral sympathetic neurons through a series of sympathetic ganglia. For purposes of the present invention, the CNS may be viewed (see FIG. 1) as consisting of a spinal cord 10 and a sympathetic trunk 12 thereof.

The PNS is shown to the right of FIG. 1 as numeral 14. The PNS is considered an automatic regulation system, that is, one that operates without the intervention of conscious thought. As such, fibers of the PNS innervate tissues in almost every organ system, providing at least some regulatory function to areas as diverse as the diameter of the eye, gut motility, and urinary output. For purposes of the present invention, the only organs so regulated by the SNS shown are lung 16, hair follicles 18, liver 20, gall bladder 22, pancreas 24, kidney and adrenal glands 26 as well as the cells which control the muscle of the blood vessel walls.

As may be noted in FIG. 1, all neurons of nerves of the SNS of interest originate in the thoracic vertebrae of the spinal cord and pass through sympathetic trunk 12 thereof. This is known as the thoracolumbar outflow of the SNS. Therein, axons of these nerves leave the spinal cord through anterior outlets/routes thereof of the sympathetic trunk 12 and, certain groups thereof, including the groups emanating from thoracic vertebrae T6 through T12 which reach celiac ganglion 28 before dispersing to various internal organs in the thoracic region of the body. There is a an important ganglion offshoot from T9 before the celiac ganglion juncture 28 which leads to the kidney and adrenal glands 26 as well a ganglion pathway from T8 that controls systemic blood vessel constriction 29 which is associated with hypertension. From these internal organs occurs a flow of axons of these respective nerves to the base of the PNS at the vagus nerve 30 shown in FIG. 1.

To reach target organs and glands, axons must travel long distances in the body, and to accomplish this, many axons relay their message to a second cell through synaptic transmission. This entails the use of a neurotransmitter across what is termed the synaptic cleft which activates further cells known as post synaptic cells. Therefrom, the message is carried to the final destination in the target organ.

Messages travel through the SNS in a bi-directional fashion. That is, so-called efferent messages can trigger changes in different parts of the body simultaneously to further the above referenced fight-or-flight response function of the SNS. It is noted that the PNS, in distinction to the CNS, controls actions that can be summarized as rest-and-digest, as opposed to the fight-or-flight effects of the SNS. Therefore, many functions of the internal organs are controlled by the PNS in that such actions do not require immediate reaction, as do those of the SNS. Included within these is the control of the adrenal glands and kidneys 26 by the SNA, as may be noted in FIG. 1.

It may thereby be appreciated that the autonomic nervous system includes both said SNS and PNS divisions which, collectively, regulate the body's visceral organs, their nerves and tissues of various types. The SNS and PNS must, of necessity, operate in tandem to create synergistic effects that are not merely an “on” or “off” function but which can better be described as a continuum of effect depending upon how vigorously each division must execute its function in response to given conditions. The PNS often operates through what are known as parasympathetic ganglia and includes so-called terminal ganglia and intramural ganglia which lie near the organs which they innervate, this inclusive of the celiac glands 29.

In summary, a change of axon activity within an internal organ is measurable at one or more of the T6 through T12 thoracic locations of the SNS and, in principle for certain organs, also at the vagus nerve 30 of the PNS, above described.

The inventor, in clinical studies, has noticed that a dysfunction of a given internal organ can be recognized by a retardation of signal strength and reduction of stability within the neurons at the T6 through T12 locations of the spinal cord. More particularly, in persons suffering from hypertension, I have found weakness and instability of neuro-transmitted signals which would normally pass from kidney and adrenal glands 26, to vertebrae T6 to T12 of the spinal cord.

Systemic hypertension is primarily due to an increase in systemic vascular resistance and not an increase in cardiac output. Hypertension is associated with impaired kidney sodium excretion, reset baroreflexes, and reset local autoregulation responses. Alterations in the renin-angiotensin-adenosterone system and sympathetic nervous system are likely to play a role in the generation and maintenance of hypertension, due to their direct effects on kidney vascular tone and sodium excretion.

It is believed that for certain organs, appropriate measurements, if taken, at vagus nerve 30 of the PNS would show a similar retardation or instability of otherwise normal signal reaching the cranial base through the nerves of the PNS.

Responsive to the above observations, I propose treatment of this instability of the internal organs, inclusive of the kidney and/or adrenal glands, by the application of appropriate electrical or electromagnetic signals through either, or both, the T6 through T12 of the SNS and at the vagus nerve of the PNS, as a means of treating abnormal kidney, adrenal gland, and vascular wall function.

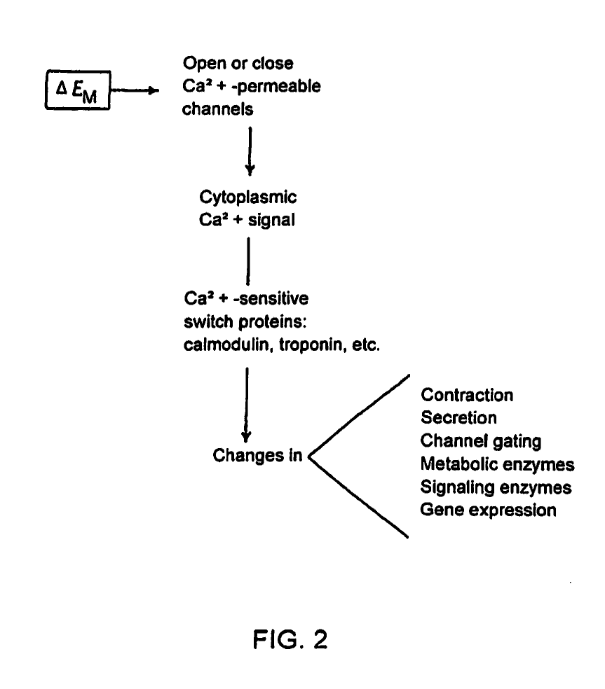

That cells of the human body are acutely responsive to electrical, magnetic and electromagnetic stimulation through neurotransmitters and otherwise, has long been established by research in the area. Calcium has been determined to be the final transmitter of electrical signals to the cytoplasm of human cells. More particularly, changes in cell membrane potential are sensed by numerous calcium-sensing proteins of cell membrane which determine whether to open or close responsive to a charge carrying elements, in this case, the calcium anion Ca²+. This is shown conceptually in FIG. 2 which shows the electrical call to action of a cell upon its sensing of a voltage gradient carried or created by a calcium anion. Stated otherwise, calcium ions transduce electrical signals to the cells through what are termed voltage-gated calcium channels (see Hille, “Ion Channels of Excitable Membranes,” 3 Ed., 2001, Chap. 4). It is now recognized that electrical signaling of voltage-gated channels (of which there are many categories) of human cell membranes is controlled by intracellular free calcium (and other) ionic concentrations, and that electrical signals are modulated by the flow of calcium anions into cytoplasm from the external medium or from intra cellular stores.

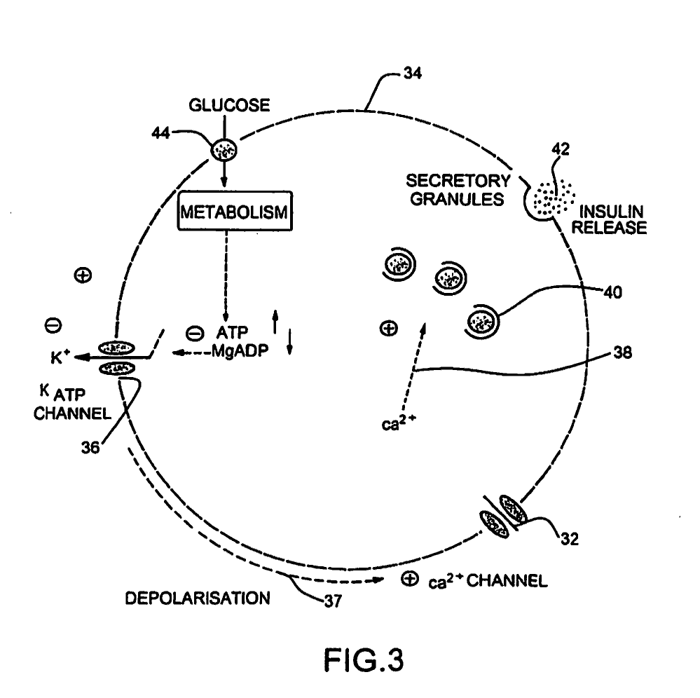

One well-studied calcium dependent process is the secretion of neuro-transmitters at nerve terminals. See Hille, page 104 thereof. Within the presynaptic terminal of every chemical synapse, there are membrane-bounded vesicular-containing high concentrations of neurotransmitter molecules of various types. When such an action potential engages a neurotransmitter, the membranes having one or more of these vesicles in their surface membrane, release a group of neuro-transmitters into the cellular space. This is conceptually shown in FIG. 2. Normally stimulated secretion from nerve terminal of most excitable cells require the extracellular calcium anions Ca²⁺ pass thru ionic channels of the cell. The above is shown at a cellular level in the schematic view of FIG. 3 which shows the calcium ionic channel 32 of cell 34 as well as the egress of a potassium anion through a so-called KATP channel 36 when a calcium anion enters the cell. This process triggers a variety of ion channel and cellular functions which relate to regulation of blood pressure as it is broadly understood.

The above principles are equally applicable to the ionic transfer function of chloride channels of the cells.

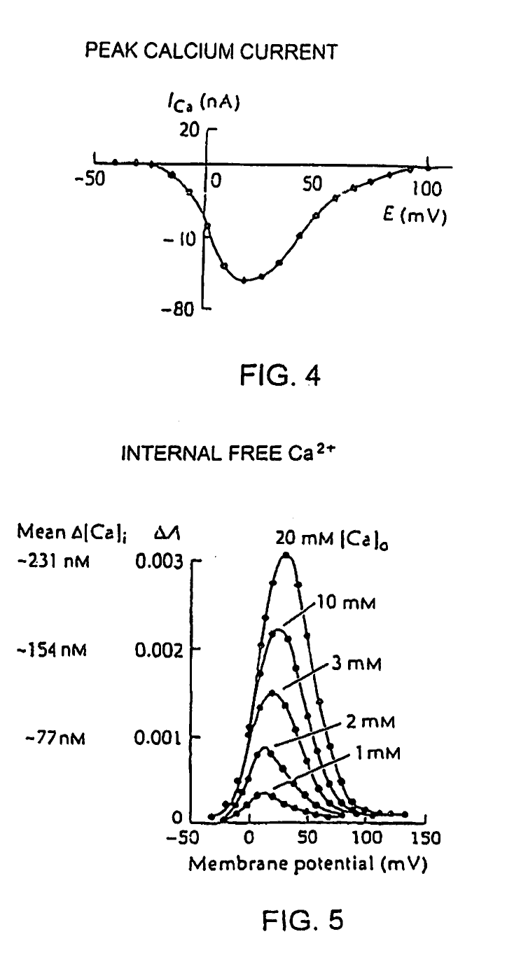

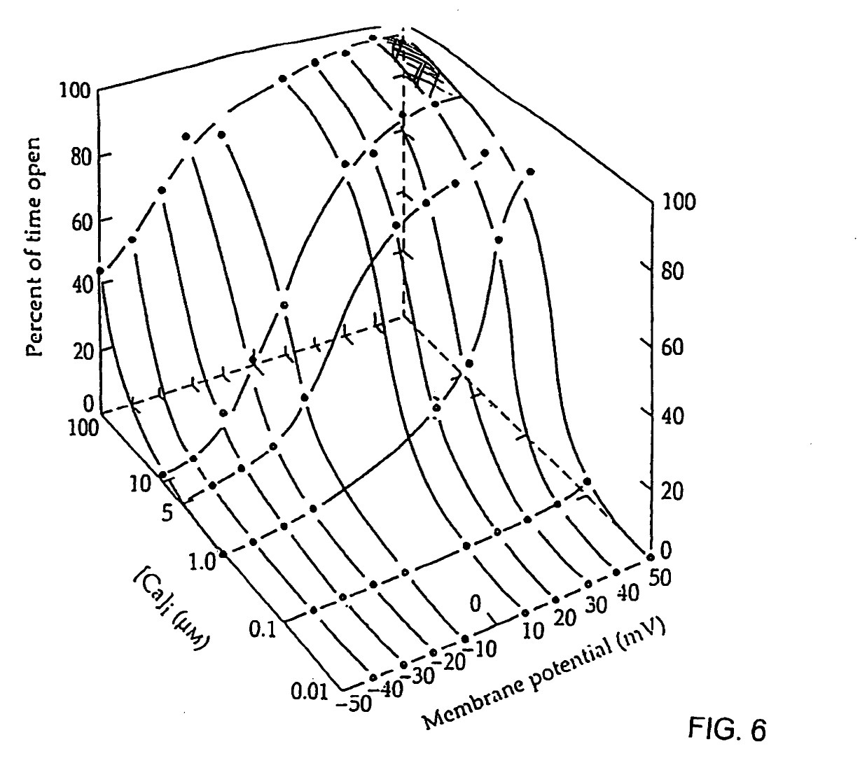

The relation of the offset of ionic calcium on membrane potential of the cell, ionic current flow within the cell, and molarity of calcium within the cell are shown in FIGS. 4 and 5 respectively. FIG. 4 indicates that the percent of time of calcium channel opening as a function of membrane potential and calcium molarity within the intracellular media. Stated otherwise, an increase in membrane potential will increase the time that voltage-gated ionic channels of the cell are open. In view of the above, it appears an appropriate increase in ionic calcium within cells of the vascular muscle cell, kidneys and adrenal glands will bring about a relaxation of the vascular muscle as well as a sodium regulation through improvement of adrenal gland and kidney function by a sufficiency of the membrane potential. The cross-hatched area at the top of the graph of FIG. 6 represents a confluence of parameters most beneficial to the health of the cell.

In view of the above, the inventor proposes the delivery of such enhanced membrane potential to cells of the vascular muscle cell, kidneys and adrenal glands through the SNS and/or PNS, as above described with reference to FIG. 1, by the application of appropriate electromagnetic signals at the T6 through T12 thoracic vertebrae and over the kidneys and adrenal glands.

Potential choices of appropriate signals may be frequency critical as has been set forth by Sandblom and George, “Frequency Response in Resonance Behavior of Ionic Channel Currents Modulated by AC Fields” 1993, who indicate that ionic channel currents calculated are frequency-dependent, describing the rates of transports of ions through channels. Liboff, et al, has proposed an optimum fluctuating magnetic field frequency for regulating transport frequency regulating transport across ionic membrane. See U.S. Pat. No. 5,160,591 (1992). The molecular characterization of the neuronal calcium channel has been studied by Perez-Ryes. Nature 1998, 391:896.

It is anticipated that appropriate electrical magnetic or electro-magnetic stimulation can be furnished to the T6 to T12, and particularly the T8 and T9 vertebrae by the use of probes, and that these would include both low and high frequency fields, inclusive of AC and DC, with AC upon a DC carrier or, as taught by Liboff above, using a Helmholz Coil to produce cyclotronic magnetic fields that are imparted to tissue or nerves of interest.

Recent developments in molecular cell biology have confirmed the principles reflected in FIGS. 2-6 above. For example, Jiang et al Rockfeller University, 2002, states: Ion channels exhibit two essential biophysical properties: a) selective ion conduction, and b) the ability to gate-open in response to an appropriate stimulus. Two general categories of ion channel gating are defined by the initiating stimulus: ligand binding (neurotransmitter or second-messenger-gated channels) or membrane voltage (voltage-gated channels), per FIGS. 4-6. The structural basis of ligand gating in a K+ channel is that it opens in response to intracellular Ca2⁺. Jiang author reports he has they cloned, expressed, and analysed electrical properties, and determined the crystal structure of a K+ channel from methanobacterium thermoautotrophicum in the (Ca2+) bound, opened state and that eight RCK domains (regulators of K+ conductance) form a gating ring at the intracellular membrane surface. The gating ring uses the free energy of Ca2+ binding to perform mechanical work to open the pore.

Many forms of cellular dysfunction have been related to the electrical call to action of cells upon sensing of the voltage gradient, the cell membrane required to open the ionic channels. As such, electrical signals are modulated by the flow of calcium anions from and to the external medium thus affecting intra-cellular storage. Correction of any malfunction in the ability of the cell to provide a proper signal is summarized in FIG. 1 and shown schematically in FIG. 2. The present invention thereby provides necessary currents and voltages, as summarized in FIGS. 3-6, necessary to optimize the flow of calcium anions to thereby restore normal function of dysfunctional cells within a given tissue. It is to be appreciated that other anions and their channels, e.g., potassium or sodium channels, may be associated with a given dysfunction.

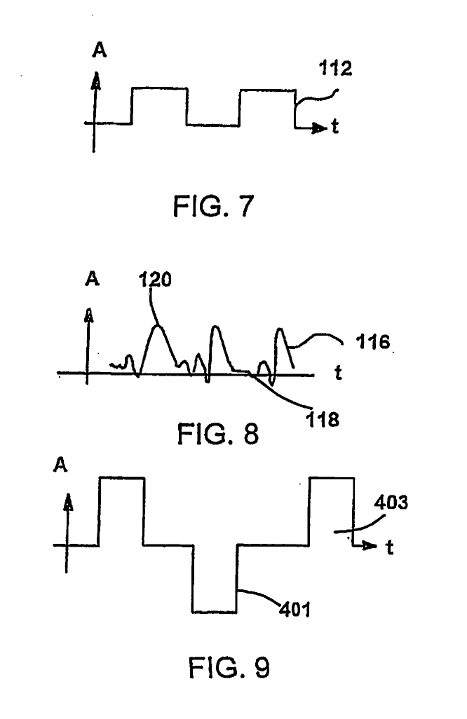

Shown in FIG. 7 is a waveform of a type used during initial probe emission 112, that is, when searching for a source of dysfunction. FIG. 8 shows a waveform that is received when a source of dysfunction is located responsive to waveform of an initial probe emission. The waveform typical of the type used at the start of treatment indicates a cell health positive response 120. However, 116 and 118 are health negative responses. The waveform of FIG. 9 is an algorithm simplified version of the waveform of FIG. 8. It includes a lower portion 401 (health negative) and upper portion 403 (health positive) which, it is to be appreciated, may be adapted in shape dependent upon the needs of a technician to better locate treatment points, such as area 403.

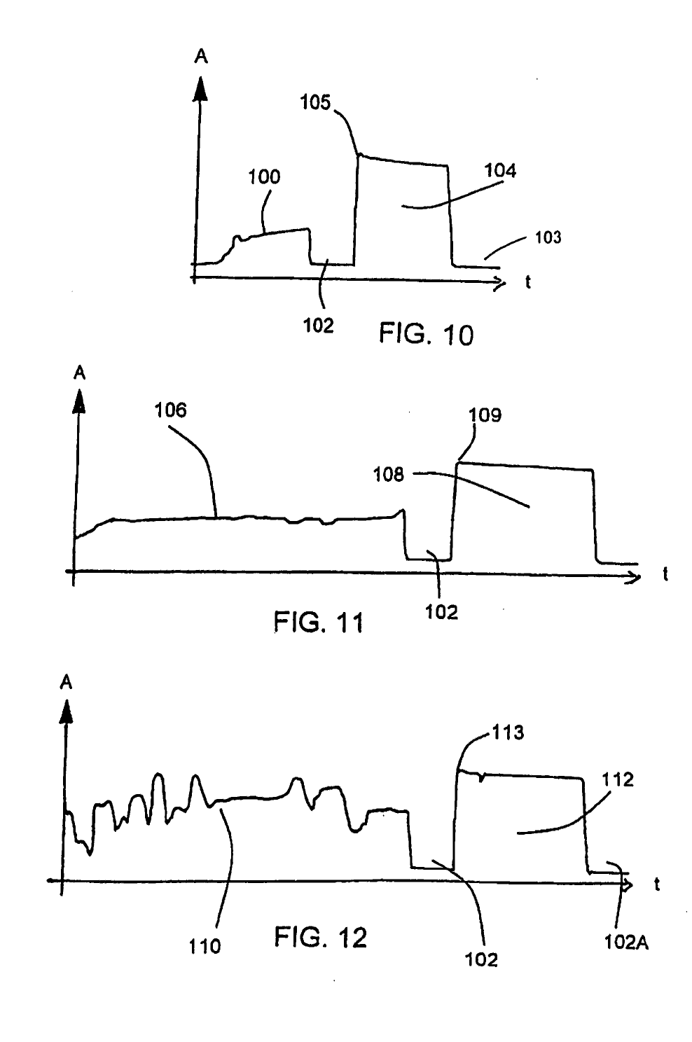

FIG. 10 is a waveform of an initial responsive following the beginning of treatment at a target site. Shown is the amplitude of a weaker segment 100 of the responsive wave, followed by transition 102 to a second segment 104 of the responsive waveform, which is a stronger or healthier response, which is followed by a further transition 103 at the right of FIG. 10. Edge 105 of waveform 104 is indicative of a higher capacitance of the part of the cell of the target site.

FIG. 11 is a view, sequential to that of FIG. 10, showing the result of initial treatment at a first site. Therein is shown that the amplitude of segment and shape of segment 100 of FIG. 10 has now increased to segment 106 of FIG. 11. This increased height waveform, as well as increased uniformity of the geometry of the waveform 106 is indicative of an induced healing process. Further is an area in which the portion 104 of FIG. 10 has changed to segment 108 shown in FIG. 11. Both segments 106 and 108 are indicative of a greater duration and length which correlates to healing at the site. Also shown is edge 109. The reduction in sharpness of edge 109 of segment 108 of the waveform indicates healing relative to the edge 105 in segment 104 of the waveform of FIG. 10.

FIG. 12 is a view at a second locus treatment of the spine showing that the treatment site exhibits a static-like and irregular segment 110 followed by a stronger segment 112 exhibiting a higher capacitance area 113. At 102 is shown a transition between segments.

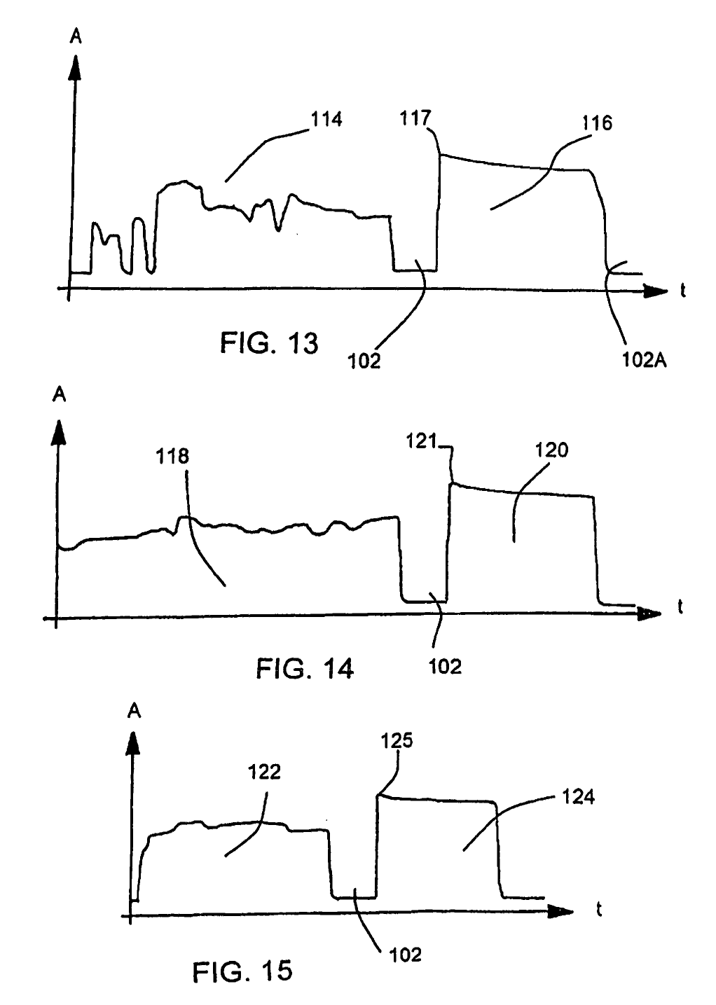

FIG. 13 is another view of the second locus of treatment within the same general therapy area. A similar pattern of static followed by a healthier area 116 is observed both upon waveforms and in an audio transform thereof (static sound versus a smooth sound). The treatment probe is moved slightly until an area of malfunction appears visually as a weak signal and, in audio, as a static or screeching sound. After a period of application of complex EM wave and energy patterns, a more positive response may be seen in FIG. 14 as much healthier segments 118 and 120, with capacitative edge 121 upon segment 120.

FIG. 15 is a waveform sequential to that of FIG. 14 in which segment 118 of FIG. 14 may be seen to be slightly changed into waveforms 122 and 124. However, segment 118 of FIG. 14 has now strengthened into a healthier waveform segment 122. Note greater the height of segment 122 versus 118. Pointed edge 125 shown in FIG. 15, is indicative of rate of change of capacitance at a treatment site, which is not desirable. Thus the waveform of FIG. 15 shows general strengthening with, however, a loss in length of the segment and a sharper edge 125 to waveform 124. Repetitive treatments of about ten minutes are needed to maximize all parameters.

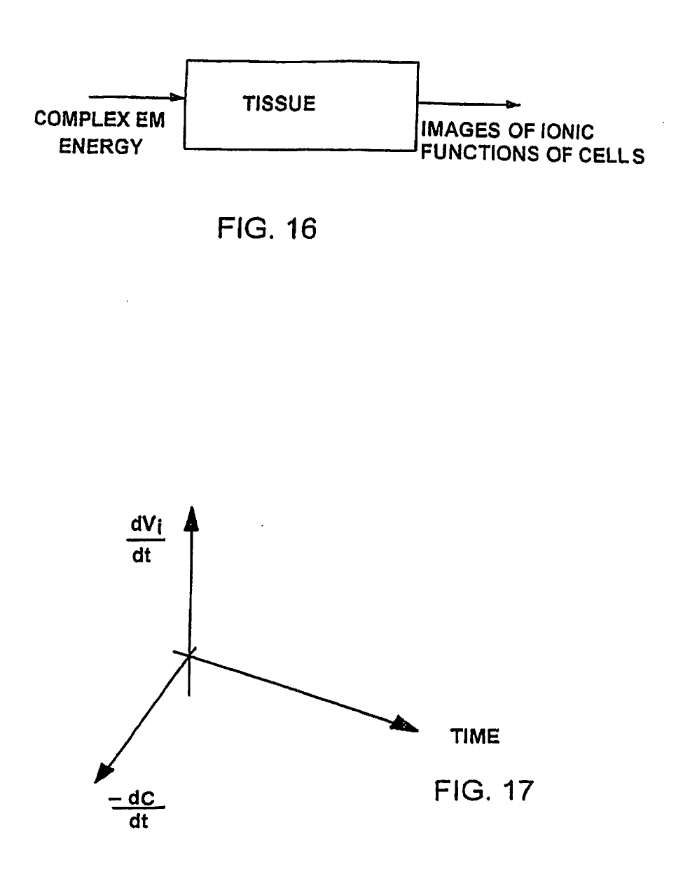

FIG. 16 is a block diagrammatic view showing how, by the input of a complex electrical and magnetic signals to a tissue site of interest, a three-dimensional image based upon a map of any selectable two of the following parameters, versus time, may be accomplished, including signal stability or rate of change in amplitude of signals. One may also calculate the first or second derivative of absolute signal amplitude as a more precise measure of signal stability. Capacitance is a further parameter that may be mapped against time to show how the effects of the treatment signal are retained at the treatment site. The derivative of capacitance may be mapped to show the rate of discharge of capacitance. Also, voltage across the cell membrane at the treatment site may, as in the view of FIGS. 4-6, be used as an important parameter, in combination with others, to produce two or three dimensional imaging of value to the treating technician and physician. The rate of change of voltage across cell membrane is also an important parameter which may be mapped both to provide a more complete picture of a user dysfunction and the result which the present therapy is effecting during treatment and between treatment session. An example of useful parameters which may be mapped in three-dimensions is shown in FIG. 17.

Example of Representative Data showing the effect of the present therapy for a single treatment is as follows:

| BP Pre | BP Post |
|---|---|
| Treatment | Treatment |
| 240/110 | 120/70 |
| 200/90 | 179/85 |
| 150/100 | 140/82 |
| 152/80 | 140/80 |

The provision of a system of electrical, electromagnetic or magnetic stimulation to one or more of the T6 thru T12 vertebrae of the human spine as well as over the kidneys and adrenal glands and, through the use of probes, imparts one or more of low frequency, high frequency, AC, DC and combinations, through the sympathetic and parasympathetic nervous systems, to appropriately regulate the activity of vascular muscle cell, kidneys and adrenal glands, to innervate such cells to better approximate normal function, inclusive of restoration of normal function from such cells of the vascular muscle, kidneys and adrenal glands and to thereby address hypertension. Vertebrae T8 and T9 are particularly applicable to this application. See FIG. 1.

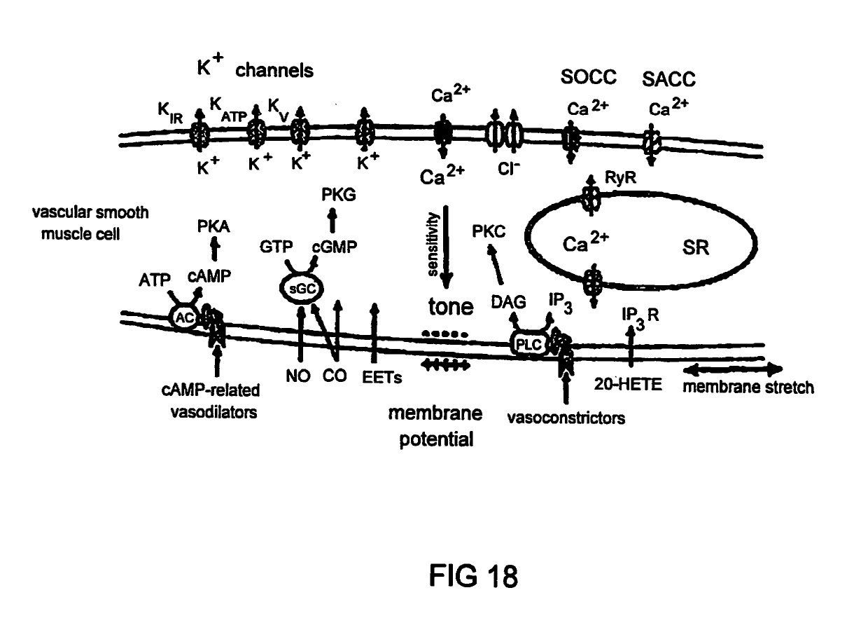

Ion channels and vascular tone. FIG. 18 is a schematic of a cross section through part of a vascular muscle cell. Along the top membrane are shown K_{IR}, K_{ATP}, K_{V}, and BK_{Ca} channels. Also shown are voltage-gated Ca²⁺ channels, 2 types of Cl⁻ channels (see text), SOC channels (SOCC), and SAC channels (SACC). Shown in the membranes of the sarcoplasmic reticulum (SR) are ryanodine receptors (RyR) and inositol 1,4,5-trisphosphate receptors (IP₃R). A few of the signals that are known to modulate the function of the ion channels depicted. AC indicates adenylate cyclase; PKA, cAMP-dependent protein kinase; sGC, soluble guanylate cyclase; PKG, cGMP-dependent protein kinase; EETs, epoxyeicostetraenoic acid (epoxides of arachidonic acid); PLC phospholipase C; DAGdiacylglycerol; PKC protein kinase C; and 20-HETE, 20-OH-arachidonic acid.

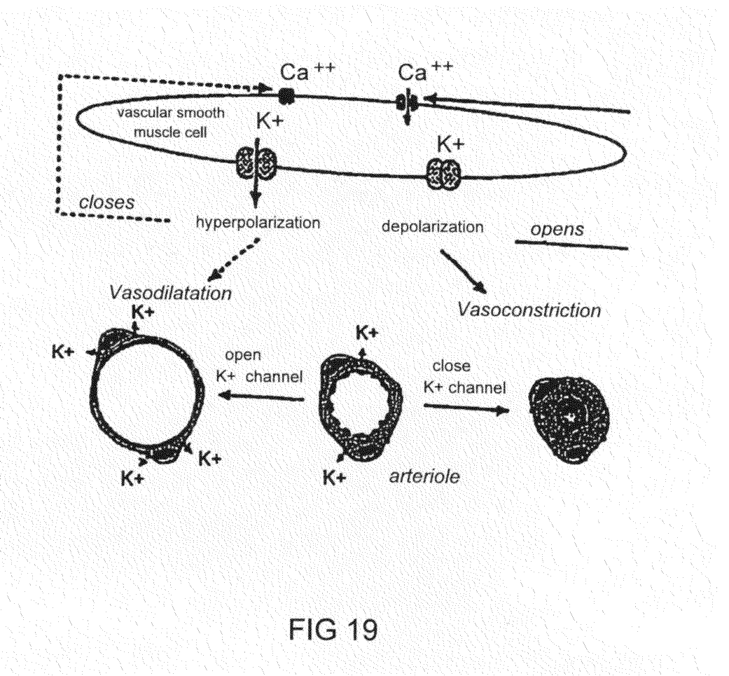

Regarding K⁺ channels and vascular tone, the schematic of FIG. 19 shows a vascular smooth muscle cell (top) and cross sections through an arteriole (bottom) that shows that opening K⁺ channels leads to diffusion of K⁺ ions out of the cell, membrane hyperpolarization, closure of voltage-gated Ca²⁺ channels, decreased intracellular Ca²⁺, which leads to vasodilatation. Closure of K⁺ channels has the opposite effect.

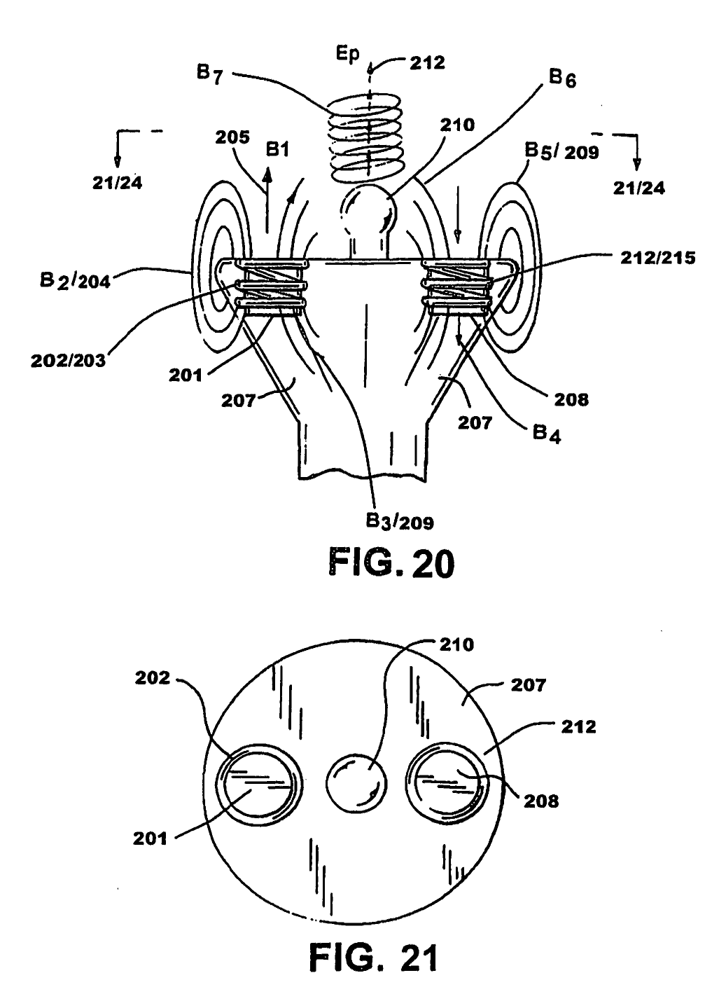

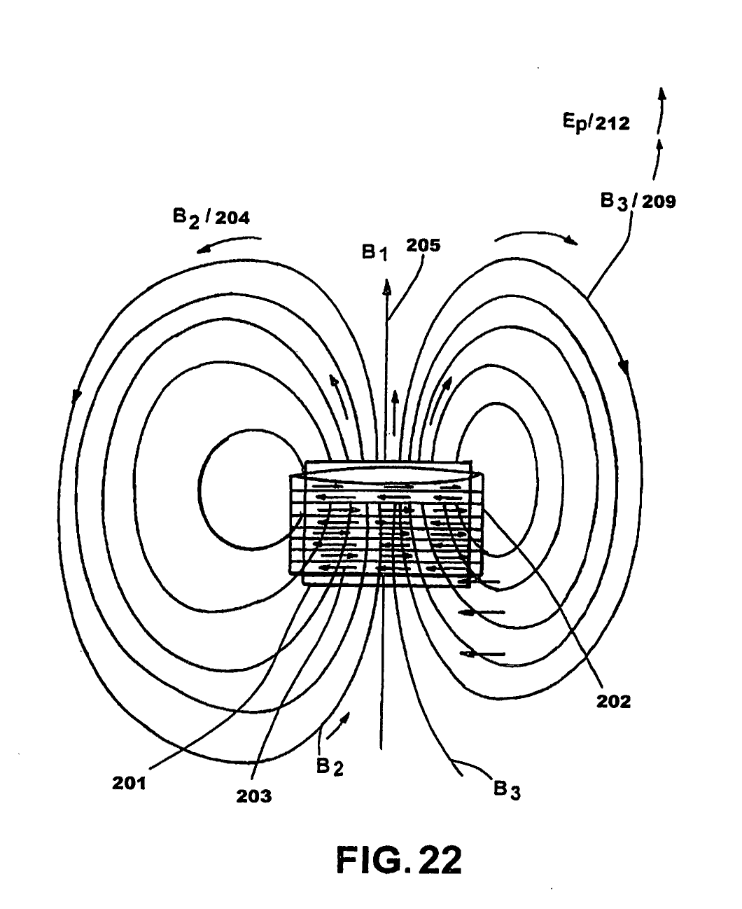

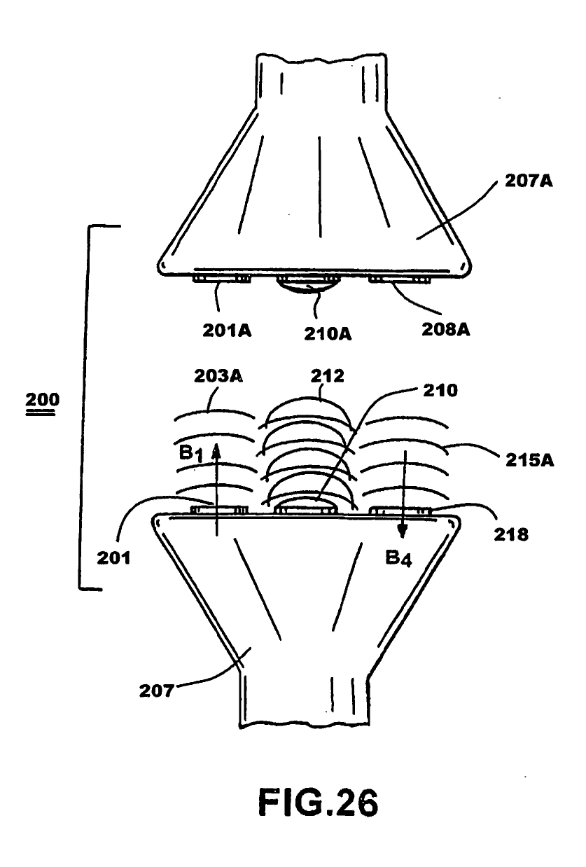

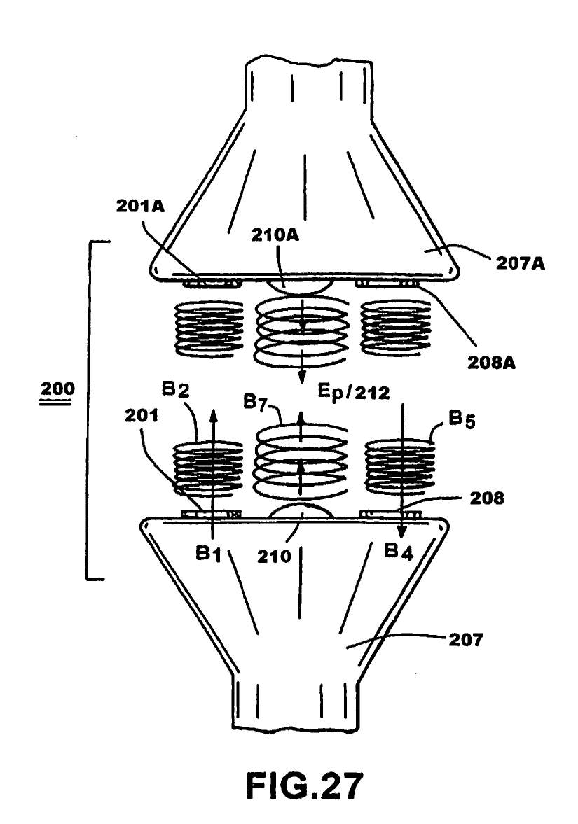

FIGS. 20, 21, 26 and 27 illustrate the general appearance of a probe 207 used in the practice of the inventive method and system of treatment of abnormalities of hypertension. The handle of probe 207 may be formed of a polymeric material such as ABS or any non-conductive equivalent thereof. Provided therein are preferably identical ferrite cores 201 and 208 around which are wound induction coils 202 and 212. Their magnetic fields may be axially variable if a pivot point for the middle of the axis of the core is provided. The axial magnetic fields resultant of these structures as shown as arrows B1 and B4 in FIGS. 20 and 21, each of which however produces oval-like peripheral outer fields B2 and B5 as well as inner fields B3 and B6 which bend in the direction of a central spherical probe 210 (see FIGS. 26 and 27) of the structure. The direction of B4 is opposite to that of B1 because the respective directions of current flow therein are opposite. Said induction coils 202 and 212 will preferably produce an inductance and associated axial magnetic fields in a range of 0.5 to 1000 milliGauss. The lateral magnetic fields B2 and B5 associated with the coils and their ferrite cores would typically fall in a similar milliGauss range. Coils 202 and 212 are powered by a current at a frequency a range of 1 to 120 G Hertz, but the current therein flow in opposite directions. See FIGS. 22 to 25.

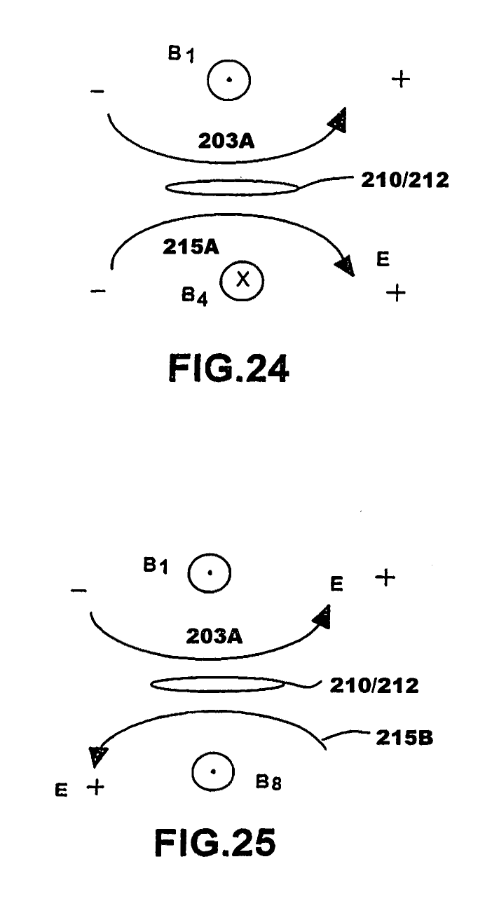

The axially disposed spherical probe 210 produces an electromagnetic pulse train Ep/212 and magnetic pulsed field B7, schematically shown as arrows and loops in FIG. 20 and as it would appear on an oscilloscope in FIG. 7, as set forth below. These AC pulses generate an associated spiral magnetic field B7 shown in FIG. 20. The primary lines of pulsed magnetic field B7 are at right angles to the primary lines of magnetic flux B1 to B4 associated with the coils 202 and 212 above described. The fact that electrical pulse 212 is projected at a right angle, particularly to fields B1 and B4, will result in a so-called ExB vector force which contributes to the therapeutic effects described herein. See also FIG. 24 which is a radial cross-section view of the E and M Fields, taken along Line 21/24-21/24, of FIG. 20

Spherical probe 210 therefore emits a complex pulsed EM wave into the treated tissue having, on one plane, the general pulse geometry shown in FIG. 22, as explained in the text below. For simplicity, aspects of the electrical signal 212 caused by the above-referenced cross-vector effect are not shown. However, it is to be appreciated that the waveform of FIG. 7 includes a magnetic component which projects transversely to the plane of the image shown in FIG. 7 prior to and during response from the tissue.

Following direct physical administration of probe 210 to soft tissue, or neuronal cells, complex respectively transverse electrical and magnetic fields will be induced into the treated tissue. This is the case whether the patient suffers from inflammation, blood loss, neurologic damage, fibrosis, devascularization, or a variety of other conditions. All will respond in a manner very generally depicted by wave forms 216/220 in FIG. 8. However, pattern segments 218 of low energy indicate a malfunction of the target tissue. Segments 220 indicate healthier cell function.

All waveforms are digitally converted to an audio transfer or color histogram for use by the system technician or clinician. Generally, the degree of static, randomness, or weakness of signal 216/218/220 is an indication of a degree of cellular or tissue level dysfunction of some type. Often, visual static will be expressed as an unstable oscillating sound in the audio transform. More particularly, if the waveform shown in FIG. 8 does not exhibit a particular degree of dysfunction, that will generally indicate to the technician that probes 207 and associated fields have not contacted the damaged or dysfunctional area of the tissue. In such case, the technician slowly positions and re-positions the probe until both the time domain and amplitude level of the static segment 218 is maximized. In a typical treatment scenario, when the probes 207 are correctly located at the cellular area most damaged or dysfunctional, extreme static will be heard through the audio transform of signal 216/218/220. When the clinician hears such high amplitude and compressed time domain static, he will enhance the level of the applied signal 212 which becomes signals 401/408 in FIG. 9. This is the so-called treatment or healing signal of the present invention, the effectiveness of which is enhanced by the various magnetic fields B1 to B7, above discussed, per FIGS. 20-27, as well as the cross-vector force (see FIG. 24) associated with the interaction of electrical and magnetic fields projecting at right angle to each other. As such, the treatment of the invention is not simply unidirectional, or one defined by the directionality of EMF field Ep/212 (see FIG. 20) but, as well, by cross-directional magnetic and ExB forces which, it has been found, enhance healing and normalization of numerous neurologic dysfunctions including, without limitation, nerve bruises, soft tissue inflammation, including joint dysfunctions particular to arthritis, all having relevance to hypertension, particularly in the T8-T9 region and its neural offshoots.

Macrophage invasion is reversed as is fibroblast proliferation, permitting revascularization and the growing of healthy new tissue. Regarding to the duration of treatment at a given treatment site, the instant protocol is to apply and increase the signal 212 or 403 to the highest level which the patient can tolerate until the response train 216 (see FIG. 8) moves above the axis stability indicating strength and stability. It has been found that after treatment with wave form 403 of FIG. 9, at the highest EMF level which the patient can tolerate, a return to normality of a particular tissue area treated, often occurs in a matter of just 10 to 15 seconds. The clinician then proceeds to locate other cells or tissue in the same area also associated with the malfunction. A few clusters of damaged cells will typically occupy a given treatment area. By searching for areas of static, as above described, the technician is able to treat damaged tissue or associated neurons to promote both healing of soft tissue and of nerve fibers. It has been found that a patient, treated three times a week for a period of about three weeks can experience substantially and permanent relief from a wide range of soft tissue and nerve-related dysfunctions.

It is to be appreciated that a goal of the product therapy is to normalize the components of the apparently random static signal (referenced above) by normalizing each of the constituent levels of dysfunction through the use of selective E and B fields and pulses, typically by an opposing E or B signal or field. These produce therapeutic induced currents, voltages and ExB forces in the tissue to be treated across the cell membranes of the treated tissue. The pulsed fields generated by the spherical probe 210 particularly the axial E field 212 component emitted by it has its greatest effect at the macro or tissue level.

The alternating B fields produced by the two lateral coils 202 and 212 will, under Faraday's Law, induce low level alternating E fields that will reach across the air gap between probes 207 and 207A (see FIGS. 26 and 27). These low level E fields, in the millivolt range, affect the action potential of the ionic channels (some of which are paramagnetic), e.g., channels of the nociceptive neurons, thus causing these channels to expel sodium anions to the outside of the cell. Excessive intra-cellular sodium is a source of pain and inflammation. The low level E field will, it is believed, also help to open the calcium anion channels by increasing the millivolt level action potential of those channels, triggering an inflow of calcium anions, which effect also causes a K anion inflow to the cell. As such, a proper balance of sodium, calcium and potassium anions between the intra- and extra-cellular fluid is accomplished, reducing pain and inflammation.

Calcium anions are also a known second messenger of many cell functions. Thereby, normalizing the intra to extra cellular balance of calcium anions operates to normalize the second messenger functions thereof.

The effect of the ExB vector force (see FIG. 24) is most likely that of a micro-vibration that operates as a micro-massage that helps to eject toxins from the target tissue.

The molecular manifestation of a disease would be seen in the smallest amplitude sinusoidal components of the static signal. At that level, disease appears as a distortion in the normal electron path or of the valance shell geometry of the molecule. Biologic molecules may be very large and complex. The lower energy effects of frequency, phase, amplitude and waveform of the various E and B induced fields function to correct these distortions of geometry of molecules of the target cells. As such, concurrent use of electrical and magnetic fields, inclusive of important interactions therebetween, maximize the healing function.

FIGS. 20-22, and 24-27 illustrate a detailed view of the inductive coil 202 and its associated fields. Therein is shown the flow of current 203 within the coil 202, as well as radial field B1 and hemispherical fields B2 and B3.

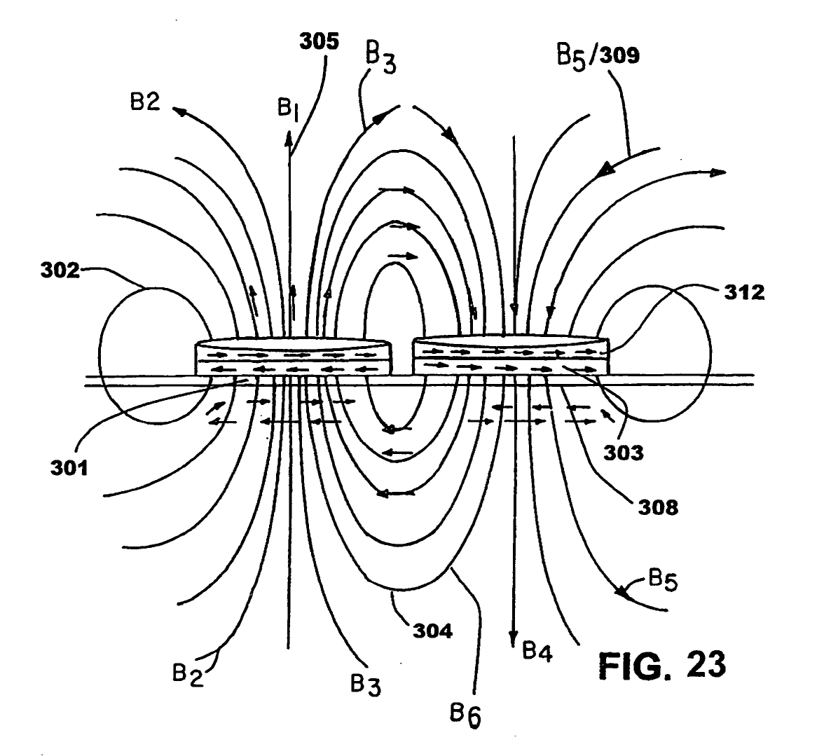

FIG. 23 illustrate an alternate embodiment 302/312 of the coils and ferrite structure of the embodiments of FIGS. 20-27. This embodiment differs from that of the previous embodiment only in the number of coils in the inductors. Such a change in the number of coil turns will produce differences in the strength and geometry of resultant magnetic fields B1 to B6. FIG. 23 also shows the continuity between field B3 of coils 301 and 311 and field B6 of said coils. Arrows inside the coils show the direction of current flow therein.

As to mechanism of operation of pulsed AC field 212 and its induced magnetic field B7 (see FIG. 20), as augmented by the above-described of magnetic fields B2-B6 of the system, it operates to influence the above-described voltage gradient associated with the calcium anions (see FIGS. 2-5) which are the final transmitter of electrical signals of human cells. Studies, as set forth in the Background of the Invention, relate the extent of passage of calcium and other anions through the ionic channels of the cell as it relates to the nerve and metabolic processes that cause many tissue and cell dysfunctions. Therein, many forms of cellular dysfunction have been related to hypertension.

Accordingly, while there has been shown and described the preferred embodiment of the invention is to be appreciated that the invention may be embodied otherwise than is herein specifically shown and described and, within said embodiment, certain changes may be made in the form and arrangement of the parts without departing from the underlying ideas or principles of this invention.

1. An EMF probe assembly for the stimulation or regulation of the T6 through T12 vertebrae and related neural offshoots, to diagnose and treat hypertension, the assembly comprising:
(a) a probe;
(b) at least one core formed of a ferro-metallic material positioned within said probe;
(c) at least one induction coil wound around said at least one core; and
(d) an interface comprising a pad for contact of said probe with or near one or more of vertebrae T6 to T12 or their neural offshoots.

2. The assembly as recited in claim 1, comprising a plurality of probes and a corresponding plurality of cores and coils thereabout in which at least one of said cores defines a sphere integral to a core at a distal end of the probe.

3. The assembly as recited in claim 2, further comprising:
an electrical pulse train furnished to a proximal end of at least one of said coils wherein a pulsed magnetic wave is thereby provided along an axis of said cores to distal ends thereof.

4. The assembly as recited in claim 3, further comprising:
a pulsed magnetic field at a distal end of said probe by furnishing an electrical current to said proximal end of said at least one coil.

5. The assembly as recited in claim 3, in which said electrical pulse train generates pulsed magnetic fields from coil at said distal end of at least one of said probes.

6. The assembly as recited in claim 5, comprising:
means for simultaneously emitting pulsed magnetic fields from said distal end of two probes of said plurality thereof.

7. The assembly as recited in claim 5, comprising:
means for simultaneously emitting a pulsed magnetic field from said spherical probe end and from one non-spherical probe end of another probe.

8. The assembly as recited in claim 7 in which a induction coils comprise:
means for generating axial fields and in combination with said sphere of one probes, hemispherical field.

9. The assembly as recited in claim 5, comprising:
means for generating a pulsed magnetic field of opposing magnetic polarity to that generated by abnormal tissue to be treated.

10. The assembly as recited in claim 5, comprising:
a pulsed electro-magnetic field, at said distal end of said distal end of at least one of said probes, having a countervailing electro-magnetic geometry to that generated by an abnormal flow of ions across a cell membrane of a given tissue.

11. The assembly as recited in claim 10, further comprising:
an audio transform for expressing electro-magnetic changes and responses of abnormal cells and tissues into human audible frequencies.

12. The assembly as recited in claim 11, further comprising:
means for adjusting said pulsed electro-magnetic fields in response to said audible frequencies.

13. The assembly as recited in claim 11, in which said audio transform comprises:
means for recognition of said responses of abnormal coils as a function of undesirable voltage gradient across membranes of cells of an affected tissue.

14. The assembly as recited in claim 12, in which said audio transform comprises:
means for recognition of said responses of abnormal coils as a function of undesirable voltage gradient across cell membrane of cells of an affected tissue.

15. The assembly as recited in claim 10, further comprising:
means for adjusting said electro-magnetic fields in response to an EM field spectrograph of a tissue abnormality.

16. The assembly as recited in claim 10, comprising:
means for viewing reactive parameters of said countervailing electromagnetic geometry.

17. The assembly as recited in claim 1, embedded within a pad or patch for contact with or near vertebrae T8 or its neural offshoots.

18. The assembly as recited in claim 1, embedded within a pad or patch for contact with or near vertebrae T9 or its neural offshoots.

19. The assembly as recited n claim 9, embedded within a pad or patch for contact with or near vertebrae T8 or its neural offshoots.

20. The assembly as recited in claim 9, embedded within a pad or patch for contact with or near vertebrae T9 or its neural offshoots.
